(19) United States
(12) Patent Application Publication
(10) Pub. No.: US 2011/0186789 A1
(43) Pub. Date: Aug. 4, 2011

(54) SYNTHESIS OF GRAPHENE SHEETS AND NANOPARTICLE COMPOSITES COMPRISING SAME

(75) Inventors: Edward T. Samulski, Chapel Hill, NC (US); Yongchao Si, Chapel Hill, NC (US); Theo Dingemans, Delft (NL)

(73) Assignee: THE UNIVERSITY OF NORTH CAROLINA AT CHAPEL HILL, Chapel Hill, NC (US)

(21) Appl. No.: 12/993,948

(22) PCT Filed: May 22, 2009
(86) PCT No.: PCT/US09/44939

§ 371 Date: Jan. 25, 2011

Related U.S. Application Data
(60) Provisional application No. 61/055,447, filed on May 22, 2008.

(51) Int. Cl. H01B 1/12; C07C 309/29; C07C 309/42; B82Y 40/00; B82Y 30/00

(52) U.S. Cl. 252/514; 562/45; 562/74; 977/896; 977/734; 977/773

(57) ABSTRACT

A method for producing isolatable and dispersible graphene sheets, wherein the graphene sheets may be tailored to be soluble in aqueous, non-aqueous or semi-aqueous solutions. The water soluble graphene sheets may be used to produce a metal nanoparticle-graphene composite having a specific surface area that is 20 times greater than aggregated graphene sheets. Graphene sheets that are soluble in organic solvents may be used to make graphene-polymer composites.

### GOVERNMENT RIGHTS

The United States Government has rights to this invention pursuant to National Science Foundation grant number CMS-0507151 and National Aeronautics and Space Administration grant number NAG-1-2301.

### FIELD

This invention relates generally to a novel method of synthesizing isolatable and dispersible graphene sheets by reducing exfoliated graphene oxide as well as the graphene sheets produced using said process. The invention further relates generally to composites comprising the graphene sheets and a method of making same.

### DESCRIPTION OF THE RELATED ART

Graphite nanoplatelets have recently attracted considerable attention as a viable and inexpensive filler substitute for carbon nanotubes in nanocomposites, given the predicted excellent in-plane mechanical, structural, thermal, and electrical properties of graphite. Graphite nanoplatelets in the form of graphene sheets are now known and each comprises a one-atom thick, two dimensional layer of hexagonally-arrayed sp²-bonded carbon atoms having a theoretical specific surface area of about 2600 m² g⁻¹. Although it is only one atom thick and unprotected from the immediate environment, graphene exhibits high crystal quality and ballistic transport at submicron distances. Moreover, graphene can be light, highly flexible and mechanically strong (resisting tearing by AFM tips), and the material's dense atomic structure should make it impermeable to gases. Graphene layers or sheets are predicted to exhibit a range of possible advantageous properties such as high thermal conductivity and electronic transport that rival the remarkable in-plane, like-properties of bulk graphite.

One possible route to harnessing these properties for potential applications is to incorporate graphene sheets in a homogeneous distribution in a composite material. As with carbon nanotubes, however, utilization of graphite nanoplatelets in the form of graphene sheets in nanocomposite applications and other applications depends on the ability to achieve complete dispersion of the graphene sheets in a solvent.

In the last few years scientists have attempted to isolate single 2D graphene sheets in a free state. This process is encumbered by the high cohesive van der Waal's energy (approximately 5.9 kJ mol⁻¹ carbon) adhering graphitic sheets to one another. One group used adhesive tape to peel off weakly bound layers from a graphite crystal, gently rubbed those fresh layers against an oxidized silicon surface, and then identified the relatively few monolayer flakes among the macroscopic shavings. (See, e.g., K. S, Novoselov et al., Science, Vol. 306, p. 666 (2004)). Another group fabricated ultrathin carbon films, typically three graphene sheets, by thermal decomposition of the surface of SiC. The SiC was simply heated sufficiently to evaporate Si from the surface, leaving behind the thin carbon films. (See, C. Berger et al., J. Phys. Chem. B, Vol. 108, p. 19912 (2004)). Jang, et al. disclosed a process to readily produce graphene sheets (U.S. patent pending, Ser. No. 10/858,814 filed Jun. 3, 2004), said process including: (1) providing a graphite powder containing fine graphite particles; (2) exfoliating the graphite crystallites in these particles in such a manner that at least two graphene sheets are either partially or fully separated from each other; and (3) mechanical attrition (e.g., ball milling) of the exfoliated particles to become nanoscaled, resulting in the formation of graphene sheets.

Disadvantageously, even assuming one is able to obtain a single 2D graphene sheet, dispersing a large number of graphene sheets in a solvent has proven to be difficult because of aggregation of the graphene sheets. Towards that end, a process of producing isolatable and dispersible graphene sheets is described herein. In addition, composites comprising the dispersible graphene sheets and a method of making same are described herein, said composites including either the isolatable and dispersible graphene sheets produced using the process described herein or alternatively, dispersible graphene sheets isolated by other means.

### SUMMARY

The present invention generally relates to isolatable and dispersible graphene sheets and methods of making and using same. The graphene sheets are functionalized and can be tailored to be dispersible in aqueous, non-aqueous and semi-aqueous solutions. One dispersed, the graphene sheets may be used to make composite materials comprising same.

In one aspect, a functionalized graphene sheet comprising a graphene sheet having at least one functional group on a basal plane of said sheet is described.

In another aspect, a functionalized graphene sheet comprising a graphene sheet having at least one functional group on a basal plane of said sheet is described, wherein the functional group comprises a sulfonic acid group and the graphene sheet is partially sulfonated.

In yet another aspect, a functionalized graphene sheet comprising a graphene sheet having at least one functional group on a basal plane of said sheet is described, wherein the functional group comprises a species selected from the group consisting of an alkyl group, an aryl group, an alkoxy group, an alkylaryl group, an alkoxyaryl group, and combinations thereof.

In still another aspect, a process of producing functionalized graphene sheets is described, said process comprising:
sonicating graphite oxide to produce exfoliated graphene oxide;
pre-reducing the exfoliated graphene oxide using a first reducing agent to produce reduced graphene oxide; and
sulfonating the reduced graphene oxide to produce partially sulfonated graphene sheets,
   wherein said first reducing agent solution is substantially devoid of ammonia, and wherein the use of polymeric or surfactant stabilizers during or after the process is not required to produce dispersible graphene sheets.

Another aspect relates to a process of producing functionalized graphene sheets is described, said process comprising:
sonicating graphite oxide to produce exfoliated graphene oxide;
pre-reducing the exfoliated graphene oxide using a first reducing agent to produce reduced graphene oxide;
sulfonating the reduced graphene oxide to produce partially sulfonated graphene sheets; and
post-reducing the partially sulfonated graphene sheets with a second reducing agent to produce partially sulfonated, dispersible graphene sheets,
   wherein said first and second reducing agent solutions are substantially devoid of ammonia, and wherein the use of polymeric or surfactant stabilizers during or after the process is not required to produce dispersible graphene sheets.

Yet another aspect relates to the further functionalization of partially sulfonated graphene sheets with at least one species selected from the group consisting of an alkyl group, an aryl group, an alkoxy group, an alkylaryl group, an alkoxyaryl group, and combinations thereof.

Still another aspect relates to a method of making a metal nanoparticle-graphene composite, said method comprising:
mixing at least one metal-containing precursor with a solvated dispersion of graphene sheets in the presence of at least one reducing agent to reduce the metal-containing precursor to a metal nanoparticle;
precipitating the metal nanoparticle-graphene sheets; and
drying the metal nanoparticle-graphene sheets to produce the metal nanoparticle-graphene composite.

In another aspect, a method of making a polymer-graphene composite is described, said method comprising:
blending graphene sheets dispersed in an organic solvent with a solution of a polymer to form a graphene-polymer mixture; and
solidifying the graphene-polymer mixture to form the graphene-polymer composite.

Other aspects, features and embodiments will be more fully apparent from the ensuing disclosure and appended claims.

### BRIEF DESCRIPTION OF THE DRAWINGS

FIG. 1 is a solid State ¹³C MAS NMR spectra (90.56 MHz; 9.4 k rpm) of graphite oxide, sulfonated graphene oxide (GO-SO₃H) and graphene; *indicates spinning side bands.

FIGS. 2 a and b are micrographs of isolated graphene oxide and partially sulfonated graphene, respectively.

FIG. 3 is a TEM image of a partially sulfonated graphene sheet.

FIG. 4 is a schematic of graphene sheets and nanoparticle-modified graphene sheets in a solvated dispersion and the dry state.

FIG. 5 is a TEM image of a platinum-graphene sheet.

FIG. 6 is an XRD diffractogram of dried graphene sheets and dried platinum-graphene composite materials.

FIGS. 7 a and b are the SEM images of dried graphene sheets and dried platinum-graphene composites, respectively.

FIG. 8 is the schematic structure of functionalized graphene.

FIG. 9 is an AFM image of functionalized graphene sheets from the dispersion in THF on freshly cleaved mica.

FIG. 10 is an ATR-FTIR spectra of functionalized graphene and water soluble graphene.

FIG. 11 is a cross-section SEM image of a graphene film prepared by evaporating a THF dispersion.

FIG. 12 is a TEM image of PMMA-graphene films containing 2 wt % graphene.

FIG. 13 is a top-surface view of a 60-70 nm thick PMMA-graphene film.

FIG. 14 is a TEM image of PEI-graphene films containing 2 wt % graphene.

### DETAILED DESCRIPTION, AND PREFERRED EMBODIMENTS THEREOF

In one aspect, a method of producing isolatable and dispersible graphene sheets is described. The graphene sheets made using said method are partially sulfonated and can be readily dispersed in water at concentrations up to about 2 mg mL⁻¹ at pH in a range from about 3 to about 10.

As used herein, the term “graphene” refers to a molecule in which a plurality of carbon atoms (e.g., in the form of five-membered rings, six-membered rings, and/or seven-membered rings) are covalently bound to each other to form a (typically sheet-like) polycyclic aromatic molecule. Consequently, and at least from one perspective, graphene may be viewed as a single layer of carbon atoms that are covalently bound to each other (most typically sp² bonded). It should be noted that such sheets may have various configurations, and that the particular configuration will depend (among other things) on the amount and position of five-membered and/or seven-membered rings in the sheet. For example, an otherwise planar graphene sheet consisting of six-membered rings will warp into a cone shape if a five-membered ring is present the plane, or will warp into a saddle shape if a seven-membered ring is present in the sheet. Furthermore, and especially where the sheet-like graphene is relatively large, it should be recognized that the graphene may have the electron-microscopic appearance of a wrinkled sheet. It should be further noted that under the scope of this definition, the term “graphene” also includes molecules in which several (e.g., two, three, four, five to ten, one to twenty, one to fifty, or one to hundred) single layers of carbon atoms (supra) are stacked on top of each other to a maximum thickness of less than 100 nanometers. Consequently, the term “graphene” as used herein refers to a single layer of aromatic polycyclic carbon as well as to a plurality of such layers stacked upon one another and having a cumulative thickness of less than 100 nanometers.

As defined herein, “substantially devoid” corresponds to less than about 2 wt. %, more preferably less than 1 wt. %, and most preferably less than 0.1 wt. % of the process solution or product, based on the total weight of said process solution or product.

As defined herein, an “alkyl” group corresponds to straight-chained or branched aliphatic C₁-C₁₀ groups. An “aryl” group corresponds to substituted or unsubstituted C₆-C₁₀ aromatic groups. An “alkoxy” group is defined as R¹O—, wherein R¹ can be the aforementioned alkyl group. An “alkylaryl” group corresponds to a molecule having both an alkyl and an aryl moiety. An “alkoxyaryl” group corresponds to a molecule having both an aryl moiety and an alkoxy moiety.

As defined herein, “non-aqueous” corresponds to a solution that is substantially devoid of added water. For example, it is understood that some chemical components naturally include negligible amounts of water. Naturally present water is not considered added water.

As used herein, the term “semi-aqueous” refers to a mixture of water and organic components.

The present invention generally relates to the functionalization of graphene sheets to produce graphene sheets that are dispersible in a solvent of choice. For example, the graphene sheets may be functionalized to be soluble in an aqueous solution or a non-polar solution.

In a first aspect, a process of producing isolatable and dispersible graphene sheets is described, said process comprising:
sonicating graphite oxide to produce exfoliated graphene oxide; and
reducing the exfoliated graphene oxide to graphene sheets,
   wherein the reduction process includes the use of at least one reducing agent, said reducing agent(s) solution being substantially devoid of ammonia, and wherein the use of polymeric or surfactant stabilizers during or after the process is not required. The graphite oxide may be purchased or may be prepared by oxidizing graphite with acid.

In one embodiment of the first aspect, the process of producing isolatable and dispersible graphene sheets comprises:
sonicating graphite oxide to produce exfoliated graphene oxide; and
reducing the exfoliated graphene oxide to graphene sheets using at least two different reducing agents,
   wherein the reducing agent(s) solution is substantially devoid of ammonia, and wherein the use of polymeric or surfactant stabilizers during or after the process is not required. The graphite oxide may be purchased or may be prepared by oxidizing graphite with acid. Preferably, a first reducing agent is used to partially reduce the graphene oxide and a second reducing agent is used to complete the reduction process later in the process.

In another embodiment of the first aspect, the process of producing isolatable and dispersible graphene sheets comprises:
sonicating the graphite oxide to produce exfoliated graphene oxide;
reducing the exfoliated graphene oxide using at least two different reducing agents and sulfonating to produce partially sulfonated graphene sheets,
   wherein said reducing agent(s) solution is substantially devoid of ammonia, the use of polymeric or surfactant stabilizers during or after the process is not required, and wherein the partially sulfonated graphene sheets are soluble in aqueous media. The graphite oxide may be purchased or may be prepared by oxidizing graphite with acid.

In still another embodiment of the first aspect, the process of producing isolatable and dispersible graphene sheets comprises:
sonicating graphite oxide to produce exfoliated graphene oxide;
pre-reducing the exfoliated graphene oxide with a first reducing agent to remove at least some oxygen functionality from the graphene oxide sheets to produce partially reduced graphene oxide;
sulfonating the partially reduced graphene oxide to produce sulfonated graphene oxide; and
post-reducing the sulfonated graphene oxide with a second reducing agent to produce partially sulfonated graphene.
   Preferably, said first and second reducing agent(s) solutions are substantially devoid of ammonia, and the use of polymeric or surfactant stabilizers during or after the process is not required, and the graphene is dispersible and soluble in aqueous media. Notably, the post-reduction process substantially completes the reduction of any remaining oxide species present on the sheets. The graphite oxide may be purchased or may be prepared by oxidizing graphite with acid. It is contemplated that the first reducing agent and the second reducing agent may be the same as or different from one another.

First reducing agents contemplated herein include, but are not limited to, alkali metal borohydrides, alkali metal cyanoborohydrides, quaternary ammonium borohydrides and amine boranes such as lithium borohydride (LiBH₄), sodium borohydride (NaBH₄), potassium borohydride (KBH₄), rubidium borohydride (RbBH₄), cesium borohydride (CsBH₄), lithium cyano borohydride (LiBH₃CN), sodium cyano borohydride (NaBH₃CN), potassium cyano borohydride (KBH₃CN), rubidium cyano borohydride (RbBH₃CN), cesium cyano borohydride (CsBH₃CN), ammonium borohydride (NH₄BH₄), tetramethylammonium borohydride ((CH₃)₄NBH₄), dimethylamino borane ((CH₃)₂NHBH₃), N,N-diethylaniline borane (C₆H₅N(C₂H₅)₂BH₃), pyridine borane (C₅H₅NBH₃), and combinations thereof. In a particularly preferred embodiment, the first reducing agent includes sodium borohydride. The first reduction process may be carried out at temperature in a range from about 60° C. to about 100° C., preferably about 70° C. to about 90° C. for time in a range from about 30 minutes to about 2 hours, preferably about 45 minutes to about 75 minutes.

Second reducing agents contemplated herein include, but are not limited to, hydrazine, 1,1-dimethylhydrazine, 1,2-dimethylhydrazine, 1,1-diethylhydrazine, 1,2-diethylhydrazine, 1-ethyl-2-methylhydrazine, 1-acetyl-2-methylhydrazine, 1,1-diethyl-2-propylhydrazine, hydrazine sulfate, sulfonated hydrazine derivatives, and combinations thereof. In a particularly preferred embodiment, the second reducing agent comprises hydrazine. The second reduction process may be carried out at temperature in a range from about 70° C. to about 130° C., preferably about 90° C. to about 110° C. for time in a range from about 10 hours to about 48 hours, preferably about 20 hours to about 28 hours. The second reduction process substantially removes any remaining oxygen functionality on the graphitic sheet.

It should be appreciated by one skilled in the art that the so-called second reducing agents may be used as the first reducing agent. In addition, when only one reducing agent is used, it may be selected from the list of first reducing agents or second reducing agents.

The partially reduced graphene oxide sheets may be sulfonated (i.e., introducing sulfonic acid (—SO₃H) groups) using any sulfonating compound under sulfonating conditions, as readily determined by one skilled in the art. For example, the sulfonating compound may be an aryl diazonium salt of sulfanilic acid or an arylalkyl diazonium salt of sulfanilic acid. The sulfonation level is stoichiometrically controlled to enable water solubility without detrimentally impacting the properties of the graphene. Notably, the introduction of sulfonate units (e.g., -p-phenyl-SO₃H) into the basal plane of the partially reduced graphene oxide prevents the graphene sheets from aggregating after the final reduction (with the second reducing agent). The sulfonation process may be carried out at temperature in a range from about 0° C. to about 20° C., preferably about 0° C. to about 5° C. for time in a range from about 30 minutes to about 4 hours, preferably about 90 minutes to about 150 minutes.

Accordingly, using the process described in the first aspect, sulfonated graphene sheets are produced that are dispersible in an aqueous solution. Accordingly, a second aspect of the invention relates to functionalized graphene sheets, wherein the functional group comprises a sulfonic acid group and the graphene sheet is partially sulfonated on its basal plane.

When the graphene sheets should be dispersible in an organic solution, the water soluble graphene sheets may be further functionalized with at least one nonpolar group selected from the group consisting of alkyl groups, aryl groups, alkoxy groups, alkylaryl groups, alkoxyaryl groups, and combinations thereof. Other functional groups may be attached depending on the end use of the graphene sheets as readily understood by one skilled in the art. Accordingly, a third aspect of the invention relates to a functionalized graphene sheets, wherein the functional group comprises a species selected from the group consisting of alkyl groups, aryl groups, alkoxy groups, alkylaryl groups, alkoxyaryl groups, and combinations thereof, and a process of making same.

For example, the partially sulfonated graphene sheets may be further functionalized using a diazotization reaction as readily understood by one skilled in the art. The extent of functionalization is stoichiometrically controlled to enable organic solvent solubility without detrimentally impacting the properties of the graphene. The process of functionalizing the graphene sheets comprises combining at least one aminated compound, water soluble graphene, a diazotizing agent, water and at least one water miscible co-solvent, and heating the reaction mixture to temperature in a range from about 30° C. to about 100° C., preferably about 50° C. to about 80° C., for time in a range from about 30 minutes to about 4 hours, preferably about 90 minutes to about 150 minutes. In a preferred embodiment, no surfactants or polymers are needed to functionalize the graphene using the diazotization reaction.

The diazotization reaction includes the generation of a diazonium salt which will subsequently attach to the basal plane of the graphene sheet. Aminated compounds are preferred for the diazotization reaction including, but not limited to, amines, diamines, aniline, or an alkyl or alkoxy derivatives thereof. The aniline derivative may include at least one alkyl group, at least one alkoxy group, or combinations thereof, wherein the alkyl and/or alkoxy groups are positioned ortho-, meta- and/or para- relative to the amine group. Aniline derivatives can include 4-(hexyloxy)aniline, phenoxyaniline, methoxyaniline, ethoxyaniline, propyloxyaniline, isopropyloxyaniline, n-butyloxyaniline, isobutyloxyaniline, sec-butyloxyaniline, tert-butyloxyaniline, 4-(heptyloxy)aniline, N-methyl-N-(2-hexyl)aniline, N-phenylaniline, 4-methyl-N-pentyl-aniline, o-ethyl aniline, p-ethyl aniline, m-ethyl aniline, o-propyl aniline, p-propyl aniline, m-propyl aniline, o-isopropyl aniline, p-isopropyl aniline, m-isopropyl aniline, o-n-butyl aniline, p-n-butyl aniline, m-n-butyl aniline, o-isobutyl aniline, p-isobutyl aniline, m-isobutyl aniline, o-t-butyl aniline, p-t-butyl aniline, m-t-butyl aniline, o-pentyl aniline, p-pentyl aniline, m-pentyl aniline, o-isopentyl aniline, p-isopentyl aniline, m-isopentyl aniline, o-s-pentyl aniline, p-s-pentyl aniline, m-s-pentyl aniline, o-t-pentyl aniline, p-t-pentyl aniline, m-t-pentyl aniline, 2,4-xylidine, 2,6-xylidine, 2,3-xylidine, 2-methyl-4-t-butyl aniline, 2,4-di-t-butyl aniline, 2,4,6-trimethyl aniline, 2,4,5-trimethyl aniline, 2,3,4-trimethyl aniline, 2,6-dimethyl-4-t-butyl amine, 2,4,6-tri-t-butylaniline, alpha-naphthyl amine, beta-naphthyl amine, o-biphenyl amine, p-biphenyl amine, m-biphenyl amine, 4-ethoxyanilne phenylethyl amine, o-methylbenzyl amine, p-methylbenzyl amine, m-methylbenzyl amine, dimethoxyphenylethyl amine, N-(2-pentyl)aniline, N-(3-methyl-2-butyl)aniline, N-(4-methyl-2-pentyl)aniline, 4-substituted aniline having the formula NH₂-phenyl-R where R═Cl, Br, I, NO₂, N(CH₃)₂, OH, COCH₃, tert-butyl, n-butyl), 1,4-bis[4-(4-aminophenoxy)phenoxy]benzene, bis[4-(4-aminophenoxy)phenyl]ether, bis[3-(4-aminophenoxy)phenyl]ether, 1,3-bis[3-(4-aminophenoxy)phenoxy]benzene, 1,2-bis(4-aminophenoxy)benzene, Bis[2-(4-aminophenoxy)phenyl]ether, 1,2-bis[2-(4-aminophenoxy)phenoxy]benzene, and combinations thereof. Preferably, the aniline derivative comprises 4-(hexyloxy)aniline or 1,4-bis(4-aminophenoxy)benzene. Other aminated compounds contemplated include, but are not limited to, straight-chained or branched C₁-C₁₀ alkylamines, substituted or unsubstituted C₆-C₁₀ arylamines, C₁-C₁₀ alkanolamines, triazoles, imidazoles, thiazoles, and tetrazoles.

Diazotizing agents include, but are not limited to, nitrite salts such as methyl nitrite, ethyl nitrite, propyl nitrite, butyl nitrite, and pentyl nitrite, or nitrous acid. In a preferred embodiment, the diazotizing agent includes isopentyl nitrite. Water miscible co-solvents can include acetonitrile, alcohol (e.g., methanol, ethanol, propanol, butanol) and acetone.

The process of producing functionalized graphene sheets that are isolatable and dispersible may further comprise centrifugation, rinsing and/or redispersion steps following the completion of the first reduction process, the sulfonation process, the second reduction process, and/or the further functionalization process, as readily determined by one skilled in the art. Preferably, when the graphene sheets are dispersible in water, the rinsing media and the redispersion media include water, preferably deionized water. When the graphene sheets are dispersible in organic solvent, the rinsing media and the redispersion media include acetone, tetrahydrofuran, 1,4-dioxane, dimethylformamide, dimethyl sulfoxide, or combinations thereof. In a further embodiment, the dispersed graphene sheets may be precipitated, rinsed and dried to produce a graphene aggregate.

The processes described herein are scalable so that large quantities of functionalized graphene sheets may be prepared which is a substantial advantage over methods known in the art.

An advantage of the processes described herein is that the functionalized graphene sheets may be tailored for dispersal on aqueous, non-aqueous, or semi-aqueous solutions. For example, the graphene sheets produced according to the processes described herein may be dispersible in water, mixtures of water and organic solvents such as methanol, acetone and acetonitrile, or organic solvents such as tetrahydrofuran, 1,4-dioxane, dimethylformamide, and dimethyl sulfoxide.

At the completion of the process of producing isolatable and dispersible graphene sheets, a novel graphene sheet exists. The water soluble graphene sheets are partially sulfonated, wherein said partially sulfonated graphene sheet has at least one of the following physical or chemical properties:
a S:C ratio in a range from about 1:35 to about 1:60, more preferably about 1:40 to about 1:55, and most preferably about 1:43 to about 1:48;
a zeta potential of about negative 55-60 mV when the pH of the graphene is about 6; the lateral dimensions of partially sulfonated graphene range from several hundred nanometers to several microns;
the partially sulfonated graphene is fully exfoliated;
the partially sulfonated graphene may be dispersed in water without the need for surfactants; and/or
the electrical conductivity is in a range from about 750 S/m to about 2000 S/m, preferably about 1100 S/m to about 1300 S/m.
   The organic solvent soluble graphene sheets have been functionalized, wherein said functionalized graphene sheet has at least one of the following chemical or physical properties:
the functionalized graphene is fully exfoliated;
the functionalized graphene can be dispersed in organic solvents without the need for surfactants; and
the lateral dimensions of functionalized graphene range from several hundred nanometers to several microns and the thickness of the sheets is about 1.5 nm.

The graphene sheets described herein may be useful in applications such as, but not limited to, composite materials, emissive displays, micromechanical resonators, transistors, ultra-sensitive chemical detectors, supercapacitors and catalyst supports.

In a fourth aspect, a metal nanoparticle-graphene composite and method of making and using same is described. The metal-graphene composite comprises metal nanoparticles adhering to the 2D graphene sheets thereby reducing the aggregation typical of graphene sheets substantially devoid of said metal nanoparticles.

As previously introduced, graphene sheets are single-atom thick sheets of hexagonally-arrayed sp²-bonded carbon atoms having a theoretical specific surface area of about 2600 m² g⁻¹. Disadvantageously, many of the properties typical of a graphene sheet devolve to that of graphite as graphene sheets aggregate and approach the 3D form of graphite. For example, solvated dispersions of graphene sheets upon drying form an irreversibly-precipitated agglomerate and the agglomerate behaves no differently than particulate graphite films with low surface areas. This degradation of the graphene properties with agglomeration would otherwise limit the potential applications of graphene in supercapacitors, batteries, fuel cells, composite materials, emissive displays, micromechanical resonators, transistors and ultra-sensitive chemical detectors.

To reduce the aggregation of graphene sheets upon drying, a metal nanoparticle-graphene composite may be produced wherein metal nanoparticles several nanometers in diameter are chemically deposited on isolated graphene sheets by reducing metal-containing precursors in solvated dispersions of graphene sheets. Although not wishing to be bound by theory, upon drying, the metal nanoparticles act as spacers inhibiting the aggregation of graphene sheets and resulting in a mechanically-jammed, exfoliated composite having a specific surface area approaching that of non-aggregated graphene sheets. This effect is illustrated schematically in FIG. 4.

In one embodiment of this aspect, a method of making the metal nanoparticle-graphene composite is described, said method comprising:
mixing at least one metal-containing precursor with an aqueous dispersion of graphene sheets in the presence of at least one reducing agent to reduce the metal-containing precursor to a metal nanoparticle;
precipitating the metal nanoparticle-graphene sheets; and drying the metal nanoparticle-graphene sheets to produce the metal nanoparticle-graphene composite.
   The mixing process may further include the introduction of at least one surfactant, at least one pH-adjusting agent, or combinations of both. The metal nanoparticle-graphene sheets may be precipitated using mineral acids such as sulfuric acid, nitric acid, and phosphoric acid.

Metals contemplated for deposition on isolated graphene sheets include, but are not limited to, Pt, Ag, Au, Cu, Ni, Al, Co, Cr, Fe, Mn, Zn, Cd, Sn, Pd, Ru, Os and Ir. Metal-containing precursors are readily contemplated in the art including metal complexes including halide (e.g., fluoride, chloride, bromide and iodide) ions, nitrate ions, sulfate ions, phosphate ions, sulfide ions, and combinations thereof. For example, when the metal to be deposited on the isolated graphene sheet includes platinum, the metal-containing precursor may include chloroplatinic acid (H₂PtCl₆). Preferably, the pH of the metal-containing precursor in water is in a range from about 4 to about 10, more preferably about 6 to about 8, and most preferably about neutral, which may be readily achieved by adding pH adjusting agent to an aqueous solution of the metal-containing precursor. The addition of neutralized metal-containing precursor minimized the aggregation of graphene sheets immediately upon addition of said precursor to the solvated dispersion of graphene sheets.

Surfactants are preferably added to the aqueous dispersion of graphene sheets containing the at least one metal-containing precursor to control the size of the metal nanoparticles and also prevent said metal nanoparticles from aggregation during reduction. Surfactants contemplated include zwitterionic betaines, wherein a zwitterionic betaine is characterized by the —OOC(CH₂)ₙN(CH₃)₂R— moiety (wherein the carboxylate has a net negative charge and the nitrogen has a net positive charge), wherein n is greater than or equal to 1 and R may be a methyl group (e.g., betaine) or some other hydrophobic tail (e.g., substituted betaine) group. Examples of zwitterionic betaine are betaine and carnitine. The related sulfobetaines and other zwitteronic surfactants with hydrophobic tails ranging from decyl to hexadecyl are also contemplated. For example, preferably the surfactant includes a sulfobetaine such as 3-(N,N-dimethyldodecylammonio) propanesulfonate. When present, a stoichiometric ratio of one (1) surfactant molecule to one (1) metal-containing precursor is preferred to inhibit metal nanoparticle aggregation during reduction although the stoichiometric range may be from 1:10 to 10:1, as readily determined by one skilled in the art.

The method of making the metal nanoparticle-graphene composite may further include the adjustment of the pH of the mixture including at least one metal-containing precursor, the solvated dispersion of graphene sheets, the reducing agent and the optional surfactant. Preferably, the pH of this mixture is in a range from about 3 to about 10, more preferably about 6 to about 8, and most preferably about neutral.

The reducing agent should not substantially aggregate isolated graphene sheets upon addition to a solvated dispersion of graphene sheets. For example, isolated graphene sheets exist in a 3:1 (v/v) water:methanol mixture, thus ensuring that the reducing agent is reducing the metal-containing precursor in the presence of substantially isolated graphene sheets.

The aqueous dispersion of graphene sheets may correspond to the graphene sheets described herein, which are soluble in water, or alternatively, other solvatable dispersions of graphene sheets may be used.

The conditions associated with the mixing of at least one metal-containing precursor with an aqueous dispersion of graphene sheets in the presence of at least one reducing agent include temperature in a range from about 60° C. to about 100° C., preferably about 70° C. to about 90° C. and time in a range from about 30 minutes to about 150 minutes, preferably about 60 minutes to about 120 minutes.

The method of making the metal nanoparticle-graphene composite may further include filtration and/or rinsing steps prior to the drying process, whereby the precipitated metal nanoparticle-graphene sheets are filtered and rinsed with a rinsing solution. The rinsing solution may include water, methanol, or combinations of both, simultaneously or sequentially.

At the completion of the process of producing a metal nanoparticle-graphene composite, a novel metal-graphene composite exists. As such, in another aspect, a metal nanoparticle-graphene composite is described herein.

In a fifth aspect, the organic solvent soluble graphene sheets described herein are blended in a polymer matrix to form a graphene-polymer composite. The process of making a graphene-polymer composite comprises blending graphene sheets dispersed in an organic solvent with a solution of a polymer, and solidifying the graphene-polymer mixture to form the graphene-polymer composite.

The term “polymer” includes homopolymers and copolymers comprising polymerized monomer units of two or more monomers. Preferred organic polymers include homopolymers, copolymers, random polymers block copolymers, dendrimers, statistical polymers linear, branched, star-shaped, dendritic polymers, segmented polymers and graft copolymers. Two or more polymers may be combined as blends or in copolymers. The polymers may be crosslinked using known crosslinkers such as monomers having at least two ethylenically unsaturated groups or alkoxysilanes. The polymers contemplated include poly(ether imide) (PEI), polystyrene, polyacrylates (such as polymethylacrylate), polymethacrylates (such as polymethylmethacrylate (PMMA)), polydienes (such as polybutadiene), polyalkyleneoxides (such as polyethyleneoxide), polyvinylethers, polyalkylenes, polyesters, polycarbonates, polyamides, polyurethanes, polyvinylpyrrolindone, polyvinylpyridine, polysiloxanes, polyacrylamide, epoxy polymers, polythiophene, polypyrrole, polydioxythiophene, polydioxypyrrole, polyfluorene, polycarbazole, polyfuran, polydioxyfuran, polyacetylene, poly(phenylene), poly(phenylene-vinylene), poly(arylene ethynylene), polyaniline, polypyridine, polyfluorene, polyetheretherketone, polyamide-imide, polysulfone, polyphenylsulfone, polyethersulfone, polyphthalamide, and polyarylamide. The polymer solutions necessary to produce said polymers are well known to those skilled in the art. Preferably, the graphene is uniformly and homogeneously distributed throughout the polymer matrix.

The graphene-polymer composites possess remarkable thermal, mechanical and electric properties and as such, may be used in the development of new coatings for use in a variety of technologies and applications.

The features and advantages of the invention are more fully illustrated by the following non-limiting examples, wherein all parts and percentages are by weight, unless otherwise expressly stated.

### Example 1

Graphite oxide prepared from natural graphite flakes (325 mesh, Alfa-Aesar) by Hummer's method was used as the starting material. In a typical procedure, 75 mg of graphite oxide was dispersed in 75 g water. After sonication for 1 hour a clear, brown dispersion of graphene oxide was formed.

The process of synthesizing graphene from graphene oxide consisted of three steps: 1) pre-reduction of graphene oxide with sodium borohydride; 2) sulfonation with the aryl diazonium salt of sulfanilic acid; and 3) post-reduction with hydrazine. In the pre-reduction step, 600 mg of sodium borohydride in 15 g water was added into the dispersion of graphene oxide after its pH was adjusted to about 9-10 with 5 wt % sodium bicarbonate solution. The mixture was maintained at about 80° C. for 1 hour under constant stirring. During reduction, the dispersion turned from dark brown to black accompanied by out-gassing. Aggregation was observed at the end of the first reduction step. After centrifuging and rinsing with water several times, the partially reduced graphene oxide was redispersed in 75 g water via mild sonication. The aryl diazonium salt used for sulfonation was prepared from the reaction of 46 mg sulfanilic and 18 mg sodium nitrite in 10 g water and 0.5 g 1N HCl solution in an ice bath. The diazonium salt solution was added to the dispersion of partially reduced graphene oxide in an ice bath under stirring, and the mixture was kept in the ice bath for 2 hours. Bubbles were expelled from the reaction mixture and aggregation was observed on the addition of the diazonium salt solution. After centrifuging and rinsing with water several times, partially sulfonated graphene oxide was redispersed in 75 g water. In the post-reduction step, 2 g hydrazine in 5 g water was added to the dispersion and the reaction mixture was maintained at 100° C. for 24 hours under constant stirring. A few drops of sodium bicarbonate solution were added into the mixture in order to precipitate the partially sulfonated graphene. After rinsing with water thoroughly, the graphene thus prepared can be readily dispersed in water via sonication.

The partially sulfonated graphene remains as isolated sheets in water after the sulfonated graphene oxide is post-reduced with hydrazine for 24 hours. In contrast, the reduction of graphene oxide with just hydrazine under similar conditions results in the formation of an irreversible aggregate and precipitate of graphitic sheets in water. The two exclusive results support the proposal that there are sulfonated units on the graphene sheets produced using the method described herein, wherein the negatively charged sulfonates (—SO₃⁻) electrostatically repel one another thus keeping the sheets separated during reduction.

Attenuated Total Reflectance (ATR) FTIR spectroscopy of the graphene sheets reveals that the oxygen-containing functional groups are substantially completely removed by the pre- and post-reduction processes, with the exception of peripheral carbonyl groups which are believed to be located on the edge of the graphene sheets and should not deleteriously impact the electronic properties of graphene.

### Example 2

The isolatable and dispersible graphene of example 1 was analyzed using solid state ¹³C Magic Angle Spinning Nuclear Magnetic Resonance (MAS NMR) spectrometry to determine the extent of graphene oxide reduction. The ¹³C MAS NMR was a Bruker 360 spectrometer operating at 90.56 MHz and used a 4 mm rotor spinning at 9.4 k rpm without decoupling.

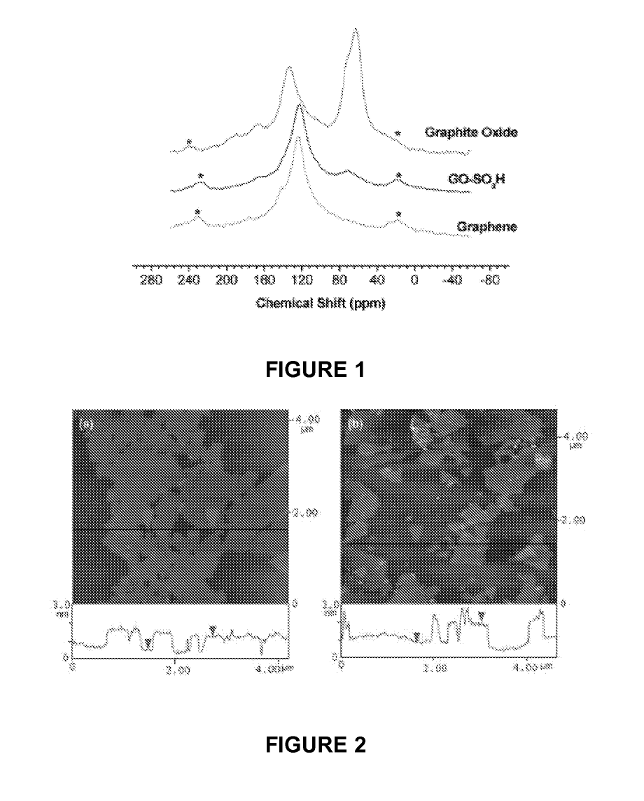

FIG. 1 shows ¹³C NMR spectra of graphite oxide, sulfonated graphene oxide (GO-SO₃H) and the graphene of example 1, respectively. Two distinct resonances dominate the spectrum of graphite oxide: the resonance centered at 134 ppm corresponding to unoxidized sp² carbons; the 60 ppm resonance is a result of epoxidation, and the 70 ppm shoulder is from hydroxylated carbons. For graphite oxide with a low degree of oxidation, the latter resonances overlap, and a weak broad resonance corresponding to carbonyl carbons is observed at 167 ppm. After pre-reduction, the 60 ppm peak disappears and the 70 ppm and 167 ppm resonances weaken significantly. The peak at 134 ppm shifts to 123 ppm due to the change in the chemical environment of the sp² carbons. After the final reduction step to yield partially sulfonated graphene, the resonances at 70 ppm and 167 ppm disappear; the small peak emerging at 140 ppm is attributed to carbons in the covalently attached-phenyl-SO₃H groups.

### Example 3

Atomic Force Microscopy (AFM) images of partially sulfonated graphene produced in Example 1 or graphene oxide on a freshly cleaved mica surface were taken with a Nanoscope III in tapping mode using a NSC14/no Al probe (MikroMasch, Wilsonville, Oreg.).

AFM images confirm that evaporated dispersions of graphene oxide and partially sulfonated graphene are comprised of isolated graphitic sheets (FIGS. 2 a and b, respectively). The graphene oxide has lateral dimensions of several microns and a thickness of 1 nm, which is characteristic of a fully exfoliated graphene oxide sheet. After the final reduction step, the lateral dimensions of partially sulfonated graphene range from several hundred nanometers to several microns. It is hypothesized that graphene sheets several microns on edge could be obtained if sonication is controlled throughout the process. The surface of the partially sulfonated graphene sheets was rougher than that of graphene oxide.

### Example 4

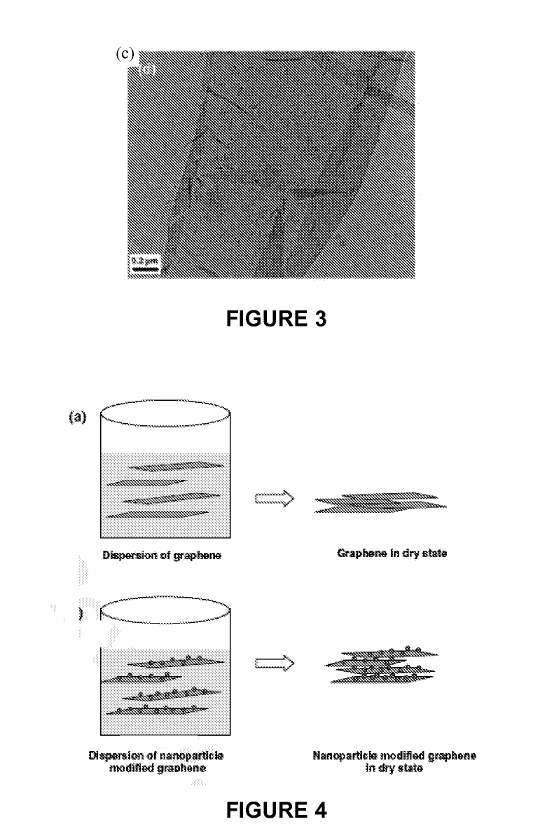

Transmission Electron Microscopy (TEM) characterization of the graphene prepared in Example 1 was performed using a transmission microscope Philips CM-12 with an accelerating voltage of 100 kV. FIG. 3 shows a TEM image of a single graphene sheet. It appears transparent and is folded over on one edge with isolated small fragments of graphene on its surface. These observations indicate the water-soluble graphene is similar to single graphene sheets peeled from pyrolytic graphite.

### Example 5

The conductivity of sulfonated graphene oxide (GO-SO₃H), the graphene prepared in Example 1, and graphite (and graphite oxide) in the form of thin films (˜3 μm thick) deposited on a glass slide was determined. The resistance of said films was measured using an Omega HHM16 multimeter (Omega Engineering, Inc., Stamford, Conn., USA). The thickness of the films was measured with a Tencor Instrument Alpha step 200 profiler (KLA-Tencor Corp., San Jose, Calif., USA). The results are shown in Table 1.

| | graphite oxide | GO—SO₃H | graphene | graphite |
|---|---|---|---|---|
| electrical | — | 17 | 1250 | 6120 |
| conductivity | | | | |
| (S/m) | | | | |

Graphite oxide is not conductive because it lacks an extended π-conjugated orbital system. After pre-reduction, the conductivity of GO-SO₃H product is 17 S/m, indicating a partial restoration of conjugation. Further reduction of GO-SO₃H to the graphene of Example 1 with hydrazine resulted in a >70-fold increase in the conductivity to 1250 S/m. By comparison, the conductivity of similarly deposited graphite flakes measured under the same conditions (6120 S/m) is only 4 times higher than that of the evaporated graphene film of the invention. The electrical conductivity of the graphene of Example 1 relative to the GO-SO₃H and the graphite suggests that much of the conjugated sp²-carbon network was restored in the graphene of Example 1, especially knowing that the lateral dimensions of the graphite flakes (30-40 microns) are more than an order of magnitude larger than the dimensions of the water-soluble graphene sheets, and lateral dimensions affect the measured conductivity.

### Example 6

Platinum nanoparticles were deposited on dispersed graphene sheets of Example 1 by the chemical reduction of chloroplatinic acid (H₂PtCl₆) with methanol in the presence of the surfactant 3-(N,N-dimethyldodecylammonio) propanesulfonate (SB 12). Specifically, 60 mg chloroplatinic acid hexahydrate (Sigma-Aldrich) in 4 g water (pH=7, after neutralized with sodium carbonate) was added into 44 g of an aqueous dispersion of graphene that contains 20 mg graphene. After 39 mg SB12 (Aldrich) in 12.5 g methanol was added into the mixture, the pH of the reaction mixture was adjusted to ˜7 with sodium carbonate. The reaction mixture was maintained at 80° C. for 90 mins under constant stirring. A few drops of dilute sulfuric acid (1N) solution were then added into the mixture in order to precipitate the Pt-graphene composite. The product was isolated by filtration, and the filtrate was colorless if all of chloroplatinic acid was reduced. After rinsing with water and methanol thoroughly, the Pt-graphene composite thus prepared was dried at 70° C. for 15 hrs.

For comparison, aggregated graphene sheets were also prepared by drying an aqueous dispersion of graphene sheets at 70° C. for 15 hrs.

### Example 7

TEM characterization of Pt-graphene composite was performed using a Philips CM-12 TEM with an accelerating voltage of 100 kV. After sonication for 5 minutes, a droplet of aqueous Pt-graphene dispersion (˜0.02 mg/mL) was cast onto a TEM copper grid followed by drying overnight at room temperature.

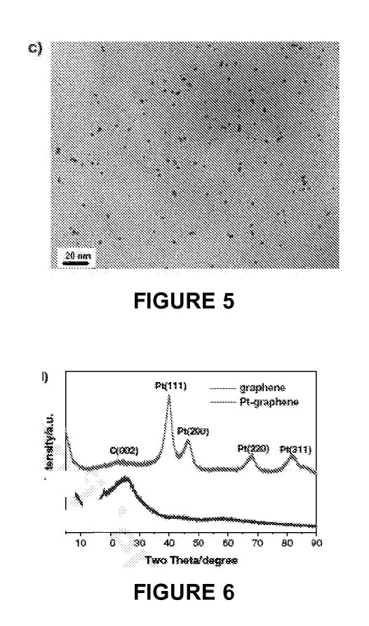

FIG. 5 shows a TEM image of platinum nanoparticles supported on graphene sheets. In this image, platinum nanoparticles appear as dark dots with a diameter of 3 to 4 nm on a lighter shaded substrate corresponding to the planar graphene sheet. The nanoparticles cover the graphene sheets with an inter-particle distance ranging from several nm to several tens of nm, occupying only a very small portion of the surface of the graphene sheet.

### Example 8

X-ray diffraction (XRD) of dried Pt-graphene (or graphene powder) was performed with a Rigaku Multiflex Powder Diffractometer with Cu radiation between 5° and 90° with a scan rate of 0.5°/min and an incident wavelength of 0.154056 nm (Cu Ka).

In FIG. 6, powder X-ray diffraction of the Pt-graphene composite exhibits the characteristic face-centered cubic (FCC) platinum lattice: diffraction peaks at 39.9° for Pt (111), 46.3° for Pt (200), 67.7° for Pt (220) and 81.4° for Pt (311) confirm that the platinum precursor H₂PtCl₆ has been reduced to platinum by methanol. The diffraction peak for Pt (220) is used to estimate the platinum crystallite size since there is no interference from other diffraction peaks. The Scherrer equation yields an average crystallite size of Pt (normal to Pt 220) on graphene of 4.2 nm, which is consistent with the TEM results. Assuming that the platinum nanoparticles are spherical, the total surface area of the composite occupied by Pt atoms was determined to be 66 m² g⁻¹.

### Example 9

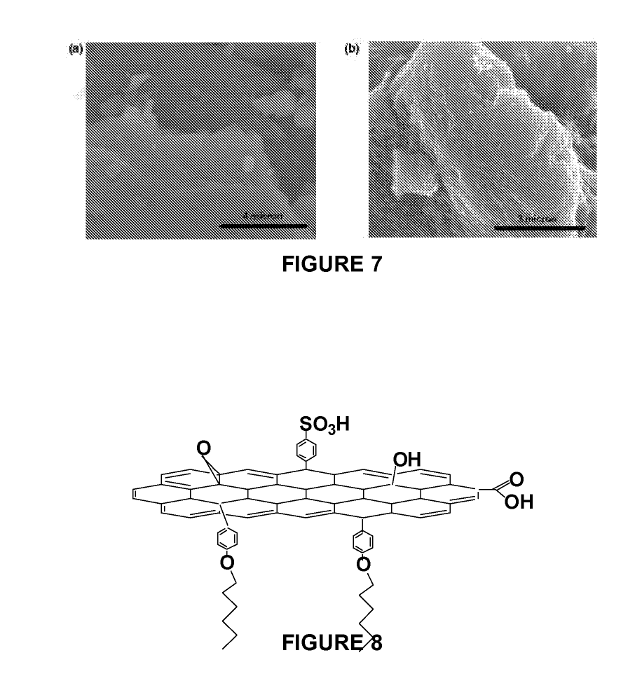

Assuming that the platinum nanoparticles of the composite of Example 6 are acting as “spacers,” the surface area of the dried platinum-graphene composite should be comparable to exfoliated graphene (i.e., graphene obtained by removing sheets of graphene from graphite). As introduced above, the theoretical specific surface area of an isolated graphene sheet should be about 2600 m² g⁻¹, so the extent of aggregation of graphene preparations can be compared to said theoretical value. For example, dried graphene sheets had a Brunauer-Emmett-Teller (BET) value of 44 m² g⁻¹, as determined using nitrogen gas absorption. In contrast, the dried platinum-graphene composite described herein had a BET value of 862 m² g⁻¹, which corresponds to an available surface area that is roughly 20 times greater than the aggregated graphene material not including platinum nanoparticles. The results suggest that the face-to-face aggregation of graphene sheets is minimized by the presence of the 3-4 nm platinum nanoparticles resulting in a jammed platinum-graphene composite. This hypothesis was corroborated by the scanning electron micrographs shown in FIGS. 7 a and b, corresponding to dried graphene sheets and dried platinum-graphene composites, respectively, wherein the dried graphene sheets of FIG. 7a appear to be fairly smooth while the dried platinum-graphene sheets of FIG. 7b appear to be much more rough. Scanning Electron Microscopy (SEM) characterization of Pt-graphene (or graphene after being dried at 70° C. for 15 hours) was performed with a FEI Helios 600 Nanolab Dual Beam System.

### Example 10

One potential application for the Pt-graphene composite is in fuel cell electrodes. In current fuel cell technology, platinum or platinum alloys are dispersed in the form of nanoparticles onto carbon black to electro-catalyze hydrogen oxidation or oxygen reduction. 2D graphene sheets promise a superior support material for a high-surface-area platinum catalyst. To that end electrodes using Pt-graphene composites were prepared and tested for oxygen reduction on the cathode in a fuel cell. The fuel cell exhibited good open-circuit voltage (˜0.99 V with H₂ on the anode and O₂ on the cathode). When the fuel cell was tested at 65° C., the cell voltage was 0.65 V at a current density of 300 mA/cm². The initial test result indicates that Pt-graphene composites are electrochemically active and catalyze oxygen reduction in a fuel cell environment.

### Example 11

Functionalization of water soluble graphene (e.g., as prepared in Example 1) was carried out with 4-(hexyloxy)aniline and isopentyl nitrite in the mixture of water and acetonitrile. For example, 52 mg 4-hexyloxyaniline (Sigma-Aldrich) and 60 mg isopentyl nitrite (Sigma-Aldrich) were added into a dispersion that contained 50 mg water soluble graphene in the mixture of 84 g water and 28 g acetonitrile under stirring. The reaction mixture was kept at 65-70° C. for 2 hours under constant stirring and graphene coagulated and precipitated in the solvents during reaction. After the product was isolated by filtration and thoroughly rinsed with acetone and THF, functionalized graphene was re-dispersed in THF to from a black dispersion after a few minutes of sonication. After rinsing thoroughly with acetone and THF, the resulting graphene would no longer disperse in water but had substantial solubility in organic solvents such as THF, 1,4-dioxane, DMF, and DMSO. The homogenous black dispersion of the functionalized graphene in THF showed good stability and no sign of coagulation after two weeks. The yield of the reaction was >90% (by wt.). The schematic structure in FIG. 8, shows the hexyloxy-phenyl functionalization along with residual oxygen functionalities present in the precursor water soluble graphene.

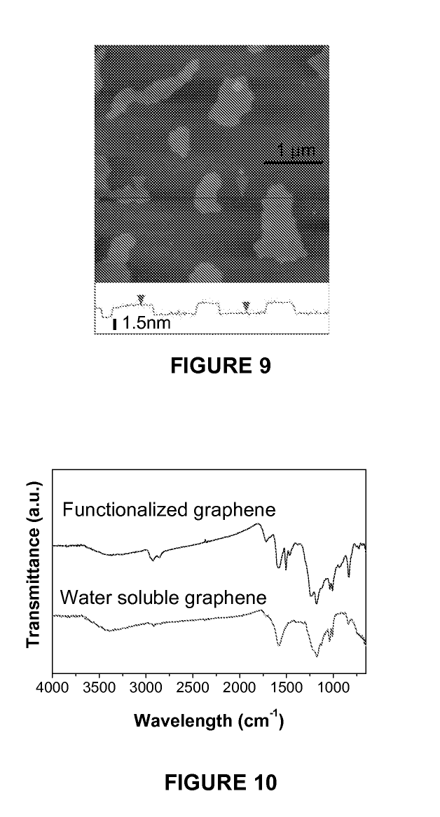

FIG. 9 shows an AFM image of graphene functionalized with 4-(hexyloxy)aniline isolated from the THF dispersion. The final graphene lateral dimensions range from several hundred nanometers up to microns; the thickness (˜1.5 nm) is slightly larger than that of exfoliated graphene and may be inflated by the presence of the functional groups. The AFM results confirm that the graphene functionalized with 4-(hexyloxy)aniline dispersed in THF is comprised of isolated graphene sheets.

Attenuated Total Reflection-Fourier Transform InfraRed (ATR-FTIR) suggests the following structural modifications of graphene functionalized with 4-(hexyloxy)aniline: the ATR-FTIR spectra (FIG. 10) illustrates the presence of the sp³ C—H stretch (2933 cm⁻¹ and 2848 cm⁻¹), the CH₃ bend (1374 cm⁻¹), an aryl ether C—O—C bond (1232 cm⁻¹), and an aromatic C═C stretch (1500 cm⁻¹ and 1470 cm⁻¹). The peak at 720 cm⁻¹ derives from the bending mode associated with four or more CH₂ groups in an aliphatic chain. None of these peaks are present in the spectrum of the precursor water soluble graphene. There is no evidence for N—H bonds (3500-3300 cm⁻¹) indicating an absence of amine groups in graphene functionalized with 4-(hexyloxy)aniline. The peak at 831 cm⁻¹ associated with the para-disubstituted phenyl group becomes more pronounced after functionalization. A broad O—H stretch band at 3500-3000 cm⁻¹ along with a C═O peak (1716 cm⁻¹) indicates the presence of carboxylic acid groups. These results suggest that p-phenyl-O—CH₂(CH₂)₄CH₃ groups were introduced into the basal plane of graphene during functionalization using 4-(hexyloxy)aniline.

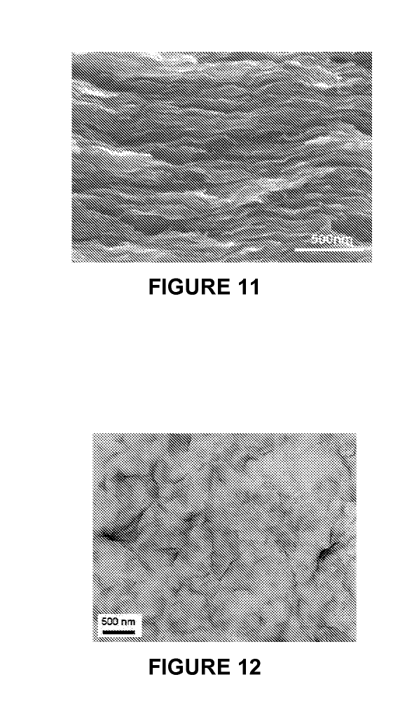

A black, freestanding graphene film (˜5 μm thick) with a metallic luster was prepared by evaporating a THF dispersion. The SEM cross-sectional image (FIG. 11), exhibits a layered morphology similar to that prepared from aqueous graphene dispersions. The film had an electrical conductivity of 125.3 S/m, indicating that a conjugated hexagonal network of sp² carbons is partially retained in the graphene functionalized with 4-(hexyloxy)aniline.

### Example 12

100 mg 1,4-bis[4-(4-aminophenoxy)phenoxy]benzene (Sigma-Aldrich) and 49 mg isopentyl nitrite (Sigma-Aldrich) were added to a dispersion that contained 20 mg water soluble graphene in a mixture of 42 g water and 14 g THF under stirring. The reaction mixture was kept at 70-75° C. for 2 hours under constant stirring and graphene precipitated in the solvents during the reaction. After the product was isolated by filtration and thoroughly rinsed with THF and NMP, the 1,4-bis[4-(4-aminophenoxy)phenoxy]benzene functionalized graphene was re-dispersed in NMP.

### Example 12

Poly(methyl methacrylate) (PMMA)-graphene composites containing 2 wt % graphene were prepared from a solution of PMMA and graphene functionalized with 4-(hexyloxy)aniline in THF after blending a dispersion of graphene functionalized with 4-(hexyloxy)aniline (4 mg/mL) in THF with a solution of PMMA (MW=350,000, Aldrich) in THF (16 wt %). After diluting with THF to the desired concentration, the mixture was sonicated for 2 hours followed by stirring with a magnetic stir bar overnight. The films were prepared by casting the PMMA-graphene mixture onto a glass slide.

Poly(ether imide) (PEI)-graphene composites containing 2 wt % graphene were prepared from a solution of 1,4-bis[4-(4-aminophenoxy)phenoxy]benzene (P3), 3,3′,4,4′-Biphenyltetracarboxylic dianhydride (BPDA) and graphene functionalized with 4-(hexyloxy)aniline in N-methylpyrrolidone (NMP) (hereinafter sample 1), a process similar to that of plain poly(ether imide). After polymerization for 24 hours under a nitrogen atmosphere, the obtained poly(amic acid)-graphene was cast onto clean glass plates. The obtained film was dried for 2 days in a N₂-purged low humidity chamber, then imidized using a convection oven. Imidization was achieved after the film was exposed to 100° C. for 1 h, 200° C. for 1 h, and 300° C. for 1 h. A second graphene-PEI polymer sample was prepared using the same method using the graphene functionalized with 1,4-bis(4-aminophenoxy)benzene (hereinafter sample 2).

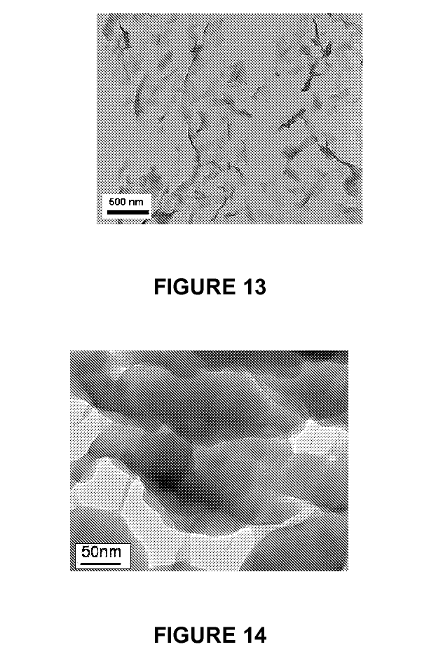

The homogeneity of PMMA-graphene composites containing 2 wt % graphene was characterized with transmission electron microscopy (TEM). FIG. 12 shows a top-view TEM image of a 60-70 nm thick film, wherein graphene sheets appear as darker shaded domains covering the whole surface. In some areas graphene sheets appear crumpled and the contour of collapsed graphene sheets is clearly seen. FIG. 13 shows a cross-section TEM image of a ˜100 nm thick PMMA-graphene film microtomed from a 60 μm thick PMMA-graphene film; the cut is approximately normal to the film surface. In FIG. 13, graphene sheets appear as darker ribbon-like areas on a lighter PMMA background. Most of ribbons have a width of 100-200 nm, and a length ranging from several hundred nanometers to over a micron, which is consistent with graphene dimensions observed in the AFM images. Sectioned, ribbon-like graphene elements were isolated from one another, indicating that there was no significant aggregation of the functionalized graphene, which evidenced a morphology having a homogeneous distribution of graphene in the PMMA matrix.

Organic soluble graphene was successfully incorporated into PEI by dispersing the organic soluble graphene in the dianhydride and diamine monomer before polymerization. TEM images of the PEI-graphene film indicates no significant aggregation of the functionalized graphene in PEI (see FIG. 14).

The two graphene-PEI polymer samples (sample 1 and sample 2) were analyzed using thermogravometric analysis (TGA), differential scanning calorimetry (DSC) and dynamic mechanical testing, as discussed below.

With regards to the TGA analysis, both samples show excellent thermal stability in N₂ at a ramp of 10° C./min. At 537° C., a 5 wt % loss was observed.

With regards to DSC, both samples had a glass transition temperature (Tg) of 210° C., followed by a large melting endotherm (max at 340° C.). Melting of the sample 1 composite was uniform while the melting of the sample 2 composite revealed two melting endotherms (overlapping) suggesting that there are two different crystal types in sample 2. In all cases the melting endotherms were observed upon successive heating and cooling, which suggests that crystallization was solvent-induced. In all cases the cooling scan and second heating scan revealed amorphous films with Tg's of ˜210° C.

With regards to the dynamic mechanical testing, both samples show an increase in r.t. E-modulus (storage modulus) from 3.4 GPa (for neat PEI polymer without graphene) to 5.5 GPa. Upon the second heat we see a moderate increase in modulus, whereby sample 1 increased to 6.4 GPa. The results demonstrate that the graphene provides a reinforcing effect.

Accordingly, while the invention has been described herein in reference to specific aspects, features and illustrative embodiments of the invention, it will be appreciated that the utility of the invention is not thus limited, but rather extends to and encompasses numerous other aspects, features and embodiments that result from the adsorption-induced tension in molecular (chemical and physical) bonds of adsorbed macromolecules and macromolecular assemblies. Accordingly, the claims hereafter set forth are intended to be correspondingly broadly construed, as including all such aspects, features and embodiments, within their spirit and scope.

1. A functionalized graphene sheet comprising a graphene sheet having at least one functional group on a basal plane of said sheet.

2. The functionalized graphene sheet of claim 1, wherein the functional group comprises a sulfonic acid group and the graphene sheet is partially sulfonated.

3. The functionalized graphene sheet of claim 1, wherein the sulfonic acid group comprises p-phenyl-SO₃H.

4. The functionalized graphene sheet of claim 2, wherein said partially sulfonated graphene sheet has at least one of the following physical or chemical properties:
a S:C ratio in a range from about 1:35 to about 1:60;
a zeta potential of about negative 55-60 mV when the pH of the graphene is about 6;
the functionalized graphene sheet is fully exfoliated;
the functionalized graphene sheet may be dispersed in water without the need for surfactants;
lateral dimensions of from several hundred nanometers to several microns; and/or
electrical conductivity in a range from about 750 S/m to about 2000 S/m.

5. The functionalized graphene sheet of claim 1, wherein the functional group comprises a species selected from the group consisting of an alkyl group, an aryl group, an alkoxy group, an alkylaryl group, an alkoxyaryl group, and combinations thereof.

6. (canceled)

7. The functionalized graphene sheet of claim 5, wherein said graphene sheet has at least one of the following physical or chemical properties:
the functionalized graphene sheet is fully exfoliated;
the functionalized graphene sheet can be dispersed in organic solvents without the need for surfactants; and/or
lateral dimensions of from several hundred nanometers to several microns and thickness of about 1.5 nm.

8. A process of producing functionalized graphene sheets comprising:
sonicating graphite oxide to produce exfoliated graphene oxide;
pre-reducing the exfoliated graphene oxide using a first reducing agent to produce reduced graphene oxide; and
sulfonating the reduced graphene oxide to produce partially sulfonated graphene sheets,
wherein said first reducing agent solution is substantially devoid of ammonia, and wherein the use of polymeric or surfactant stabilizers during or after the process is not required to produce dispersible graphene sheets.

9. The process of claim 8, wherein the pre-reduction partially reduces the graphene oxide.

10. The process of claim 8, further comprising post-reducing the partially sulfonated graphene sheets with a second reducing agent to produce partially sulfonated, dispersible graphene sheets, wherein said second reducing agent solution is substantially devoid of ammonia.

11. (canceled)

12. (canceled)

13. The process of claim 10, wherein the post-reduction process substantially completes the reduction of oxide species not reduced during the pre-reduction process.

14.-20. (canceled)

21. The process of claim 8, wherein the partially sulfonated graphene sheets are soluble in water.

22. The process of claim 8, further comprising functionalizing the partially sulfonated graphene sheets with at least one species selected from the group consisting of an alkyl group, an aryl group, an alkoxy group, an alkylaryl group, an alkoxyaryl group, and combinations thereof.

23. The process of claim 22, wherein the functionalization comprises combining the partially sulfonated graphene sheets with at least one aminated compound, a diazotizing agent, water and at least one water miscible co-solvent under diazotization conditions to produce functionalized graphene sheets.

24.-26. (canceled)

27. The process of claim 22, wherein the functionalized graphene sheets are soluble in organic solvents.

28. A method of making a metal nanoparticle-graphene composite, said method comprising:
mixing at least one metal-containing precursor with a solvated dispersion of graphene sheets in the presence of at least one reducing agent to reduce the metal-containing precursor to a metal nanoparticle;
precipitating the metal nanoparticle-graphene sheets; and
drying the metal nanoparticle-graphene sheets to produce the metal nanoparticle-graphene composite.

29. The method of claim 28, wherein the mixture including at least one metal-containing precursor, the solvated dispersion of graphene sheets, and at least one reducing agent further comprises at least one surfactant, at least one pH-adjusting agent, or combinations thereof.

30. The method of claim 28, wherein the metal nanoparticle-graphene sheets are precipitated using mineral acids.

31. (canceled)

32. (canceled)

33. The method of claim 28, wherein the at least one reducing agent comprises methanol.

34.-36. (canceled)
